(19) United States
(12) Patent Application Publication
(10) Pub. No.: US 2004/0064182 A1
(43) Pub. Date: Apr. 1, 2004

(54) High-myopia anterior chamber lens of one-piece, foldable construction

(76) Inventor: Charles David Kelman, Boca Raton, FL (US)

Correspondence Address: DARBY & DARBY P.C., Post Office Box 5257, New York, NY, 10150-5257, US

(21) Appl. No.: 10/256,214

(22) Filed: Sep. 26, 2002

(51) Int. Cl. A61F 2/16

(52) U.S. Cl. 623/6.17; 623/6.4; 623/6.54

(57) ABSTRACT

A high-myopia intraocular lens (IOL) for anterior-chamber implantation is constructed as a single, integral unit with an optic portion in the shape of a substantially circular concave lens, an annular flange portion extending radially outwards from the circumference of the optic portion, and haptic elements for fixating the IOL in its implanted position. The annular flange is substantially impervious to light and has an outside diameter at least as large as the aperture diameter of a dilated pupil of the eye. The circumferential surface of the optic portion is substantially light-impervious as well as non-reflective. The IOL is at least in part foldable to facilitate surgical implantation in the eye through a small incision.

### BACKGROUND OF THE INVENTION

The present invention generally relates to an intraocular lens (IOL) and, more specifically, to a kind of IOL that:

is used to correct myopia of, e.g., −10 to −25 diopters,

is surgically implanted in the anterior chamber through a small incision in the cornea, and

is designed to cover the pupil when the latter is dilated, so as to avoid a sensation of glare.

The surgical implantation of an intraocular lens is a well known technique that is widely used for restoring vision after cataract surgery. The cataracted natural lens of the patient is removed through a minimum size incision in the wall of the cornea of the eye and replaced with an artificial intraocular lens. Another application of IOL implants is for the correction of severe myopia, in which case the natural lens can be left in place. In all cases, it is important that the incision be made as small as possible in order to minimize the possibility of injury to the eye.

However, the size of the incision is generally dictated by the size of the artificial lens which is to be implanted. To allow for proper nighttime vision, the artificial lens should ideally be as large as the aperture of the pupil when it is dilated at low ambient light conditions. As a further consideration, the artificial lens should not cause any glare. The wearer of an IOL will have a sensation of glare if unfocussed stray light is allowed to reach the retina, as will be the case when the pupil is dilated to a larger diameter than the diameter of the IOL. The stray light can be light that passes entirely outside the perimeter of the IOL as well as light that is reflected or transmitted at the perimeter of the IOL.

Intraocular lenses for high-myopia patients have high negative diopter values, i.e., they are concave lenses. This creates a unique problem associated only with anterior-chamber high-myopia IOLs: as the diameter of the IOL is increased so as to avoid glare conditions, there is a danger that the thickened peripheral portion will come into contact with the inner surface of the cornea, resulting in potential injury. To avoid this problem, the diameter of the optic or lens body may be reduced in order to avoid the above-mentioned peripheral contact. However, in this latter situation, the optic will be smaller than the pupil in its dilated condition, resulting in glare caused by unfocussed stray light as explained above.

Many state-of-the-art IOLs are foldable to allow their implantation into the eye through a minimum size incision. After the folded IOL has been passed through the incision, it unfolds or expands to its final size and shape inside the eye.

Also of concern with IOLs is their biocompatibility. Hydrophilic materials such as hydrophilic acrylics and hydrogels have been found to be particularly tissue-friendly materials for use in IOLs.

A solution meeting at least the most essential requirements applicable to high-myopia IOLs is disclosed in my U.S. Pat. No. 5,769,889, which is incorporated herein by reference. The IOL described therein is a two-piece anterior chamber artificial intraocular lens for treating high myopia conditions by implantation in an eye, e.g., after removal of the natural eye lens, or in cases where the natural eye lens is left intact. The two-piece assembly is inserted through a minimum size incision in the eye. The lens includes a lens body or optic and a separate ring-shaped tension frame surrounding the optic at the perimeter and containing light masking means for blocking light rays from reaching the outer edge portions of the lens body where they could be scattered toward the retina and cause the sensation of glare that has been described above.

Further in the state of the art according to U.S. Pat. No. 5,769,889, the lens body or optic is generally circular and has a maximum diameter of approximately 3.5 to 5.0 millimeters. The lens body, or optic, is conveniently made of shape-retaining plastic. The optic, which is generally smaller than the diameter of a pupil dilated for night vision, is surrounded by a snugly fitting opaque or semi-opaque ring or frame having a C-shaped cross section and a peripherally extending fin or flange of the same material. The lens is also provided with haptics (position fixation means), which are integrally formed with the lens body and extend outward in the generally horizontal plane of the lens body for seating the lens in the eye. The haptics and lens body are preferably made of polymethylmethacrylate (PMMA).

The frame is a generally ring-shaped element with a channel profile that is open towards the center of the ring. The channel holds the outside border of the lens body. Integrally formed with the channel of the frame is a thin, preferably annular fin or flange extending radially outward from the channel profile. The frame has radially extending notches or slots through which the haptics pass from the lens body to the outside of the frame. The channel profile and annular fin are preferably formed of, or coated with, optically opaque (non-translucent and non-reflective) material in order to function as light masking means. The frame is preferably made of silicone and is snapped onto the optic during manufacturing. During insertion into the eye, the flexible fin is folded or bent so as to facilitate implantation of the assembled IOL unit into the eye through a minimal-size corneal incision. Once the IOL unit is inserted into the eye, the fin returns to its original radially outwardly extending position.

However, while the IOL of the foregoing description according to my earlier U.S. Pat. No. 5,769,889 meets the most essential requirements, some of the particular attributes that have been identified above as desirable in high-myopia IOLs are not being realized to the fullest extent: With the 2-piece construction as disclosed therein, only the fin of the frame and the haptics are foldable, requiring an incision large enough to allow insertion of the unfolded optic. The frame embracing the optic portion around the top and bottom adds to the thickness at the periphery of the optic, conflicting to some extent with the objective of avoiding contact with the cornea. The radial width of the frame detracts from the effective lens surface area available for night vision with dilated pupils. Also, the possible benefits of using hydrophilic materials for better tissue compatibility are not addressed in U.S. Pat. No. 5,769,889.

### OBJECT OF THE INVENTION

The present invention therefore has the object of providing a myopia or even hyperopia anterior-chamber lens that overcomes the drawbacks and inefficiencies of the prior art and incorporates to the fullest extent possible the aforenamed desirable attributes, i.e., that the IOL a) can be inserted into the eye through a minimum-size incision, b) avoids contact with the cornea, c) prevents glare, d) affords the best possible level of night vision, and e) is made of tissue-friendly materials.

### SUMMARY OF THE INVENTION

According to the present invention, a high-myopia intraocular lens that meets the foregoing objectives is monolithically constructed as a single, integral body, rather than being an assembly of a plurality of parts as is the case with the state-of-the-art IOL described above. The single-piece IOL of the present invention has a substantially circular optic portion. According to the nature of high-myopia cases, the optic portion has a negative diopter value and is therefore thickest at the periphery and thinnest at the center. To avoid contact with the inside of the cornea and also to allow insertion through a small incision, the optic portion has to be smaller than the aperture of the fully dilated pupil of the eye. Surrounding the optic portion along its perimeter, the IOL has an annular flange portion extending substantially in a plane in an outward radial direction from the optic portion. The annular flange has an outside diameter at least as large as the aperture diameter of a dilated pupil of the eye. As a means of fixating the IOL inside the eye, the IOL has so-called haptics extending substantially in an outward direction from the optic portion. At least the annular flange portion and the haptic portion have sufficient flexibility so that they can be folded towards the center during the surgical insertion and return to an unfolded state after they have been passed through the incision. While at least a central area of the optic portion is substantially transparent, the flange portion covers a sufficient area and is sufficiently opaque so that little or no light passing outside the optic portion can enter the pupil even when the latter is fully dilated. For best results, the substantially cylindrical side wall or circumference of the optic portion, likewise, should be made impervious to incident light that enters the eye at an oblique angle and falls on the cylindrical side wall of the lens body. Furthermore, the circumferential side wall should be non-reflective so that no light can be reflected inside the optic portion. All of the aforementioned measures are designed to let light enter the pupil and reach the retina only on the intended refractory path through the optic portion, so that any sensation of glare due to stray light is avoided.

It should be noted that terms such as “sufficiently opaque” or “substantially impervious” as used herein are meant to specifically imply that the invention is not limited to solutions requiring a surface coating containing a dye, but includes solutions where the elements characterized as “sufficiently opaque” or “substantially impervious” are dispersing light, e.g., because they have a dull or rough surface or a milky or turbid consistency.

High-myopia lenses are by their nature of a shape where the anterior outside edge of the lens is closest to the inner surface of the cornea. In a lens which, to begin with, is as thin as its concavity will allow, the risk of contact between the edge of the lens and the curved interior surface of the cornea can be further minimized by reducing the diameter of the lens. According to the invention, the implanted intraocular lens is designed to avoid contact between the lens and the inner corneal surface, e.g., by a suitable choice of the thickness and diameter of the lens.

Preferably, an intraocular lens according to the invention is made of a hydrophilic material, because such materials have been proven to be well tolerated by wearers or IOLs, so that the risk of inflammation is minimized. The hydrophilic material should be capable of holding a water content of at least 20 percent, the preferred range being between 20 and 30 percent water content. Recommended hydrophilic materials are found among acrylic polymers and hydrogels.

Nevertheless, other proven state-of-the-art materials for IOLs such as PMMA (polymethylmethacrylate) or certain silicone materials could also be considered for single-piece IOLs. Furthermore, it should be understood that a single-piece design does not necessarily require all parts of an IOL to be made of the same material. It is conceivable, for example, that the optic portion, the flange portion and the haptic portion are made of different materials and bonded together into a single, integral body, e.g., by ultrasonic welding or any other connection method known in the art. This would allow the use of a material with the most desirable properties for each individual portion of the IOL such as, for example, a high refractive index for the optic portion combined with an ideal degree of flexibility for the haptic and flange portions.

The preferred range for the diameter of the optic portion is between 3.5 and 5.0 millimeters, and the flange portion should preferably be about 0.3 to 1.0 millimeters wide in the radial direction of the IOL.

The flange portion as well as the circumference of the optic portion can be made impervious to incident light by chemical or mechanical treatments, or also by an appropriate surface coating of the flange portion and/or the circumference of the optic portion. The treatment should be of a kind that also makes the circumference of the optic portion non-reflective, so that no light reflection can take place inside the optic portion.

The flange portion preferably extends in a plane that runs parallel to the main plane of the optic member, closer to the posterior face than to the anterior face of the lens by a predetermined distance in the range of 0.5 to 1.0 millimeters.

### BRIEF DESCRIPTION OF THE DRAWINGS

Other objects, features and advantages of the invention discussed in the above brief explanation will be more clearly understood when taken together with the following detailed description of an embodiment which is meant to be illustrative only, and the accompanying drawings reflecting aspects of that embodiment, in which:

FIG. 1 represents a plan view of an embodiment of an intraocular lens according to the prior art as described in my U.S. Pat. No. 5,769,889;

FIG. 2 represents a cross-sectional view of the intraocular lens of FIG. 1 taken along the line A-A;

FIG. 3 represents a plan view of an embodiment of an intraocular lens according to the invention;

FIG. 4 represents a cross-sectional view of the intraocular lens of FIG. 3 taken along the line B-B; and

FIG. 5 schematically illustrates an IOL of the present invention implanted in the anterior chamber of an eye.

### DETAILED DESCRIPTION OF THE PREFERRED EMBODIMENTS

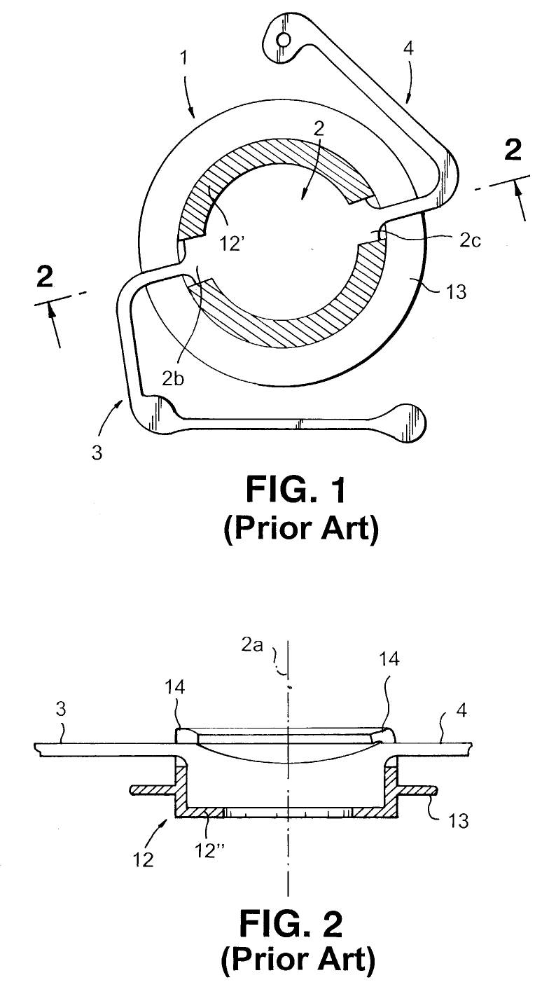

FIGS. 1 and 2 serve to illustrate the essential features of the prior art according to U.S. Pat. No. 5,769,889, which forms the background to the present invention. An anterior-chamber intraocular lens 1 has a central optic portion 2 with an optical axis 2a (shown in FIG. 2). A first fixation element 3 and a second fixation element 4 extend outwards from approximately opposite parts 2b and 2c of the periphery of the optic portion 2. The fixation elements 3, 4 run in partially radial and partially tangential directions, generally in an opposite sense with respect to each other.

The fixation elements 3, 4 (also called haptics) are designed to hold the lens fixed at three points. One or both of the fixation elements 3 and 4 are resilient i.e., springy, such that they will return to the original undeformed condition shown in FIG. 1 after they have been bent or folded away from the illustrated configuration.

FIG. 2 gives a cross-sectional view of the same stateof-the-art intraocular lens 1 as illustrated in FIG. 1. As can be seen with particular clarity in FIG. 2, the IOL according to U.S. Pat. No. 5,769,889 has a ring-shaped frame 12 surrounding and holding a lens body 2. The lens body 2 is designed for patients suffering from high myopia, i.e., as a concave lens. Frame 12 is generally circular and has inward-protruding rims 12′ and 12″ to secure the lens in the frame. The rim 12′ has slots 14 for the haptics 3, 4. An annular fin 13 is formed integrally on the outside perimeter of the frame 12, extending radially outward. In the state-of-the-art IOL, the optic 2 and the haptics 3, 4 can be manufactured as a unit, of a single piece of polymethylmethacrylate (PMMA), or a similar biologically inert plastic material, while the frame 12 is formed as a separate part, preferably of a silicone material. The function of the frame 12 is to eliminate or at least reduce glare. As mentioned previously, a glare effect occurs if reflected light from the cylindrical perimeter wall of the optic 2 or incident light entering outside the perimeter of the optic is allowed to reach the retina. To achieve this effect, frame 12 is preferably formed of substantially opaque material in order to function as a light masking means at the periphery of the lens body 2.

FIG. 2 clearly illustrates two major drawbacks of the prior art according to the aforementioned U.S. Pat. No. 5,769,889, namely:

The frame 12 adds to the diameter and thickness at the periphery of the lens body 2. Thus, to accommodate a lens body 2 with a frame 12 inside the curvature of the cornea, the lens body 2 needs to be made smaller than for a lens without the frame 12.

The effective lens diameter is further reduced by the inward-protruding rims 12′, 12″ of the channel profile by which the frame embraces the lens body. As mentioned previously, both of the foregoing factors cause a reduction of the effective lens surface area available for night vision with dilated pupils.

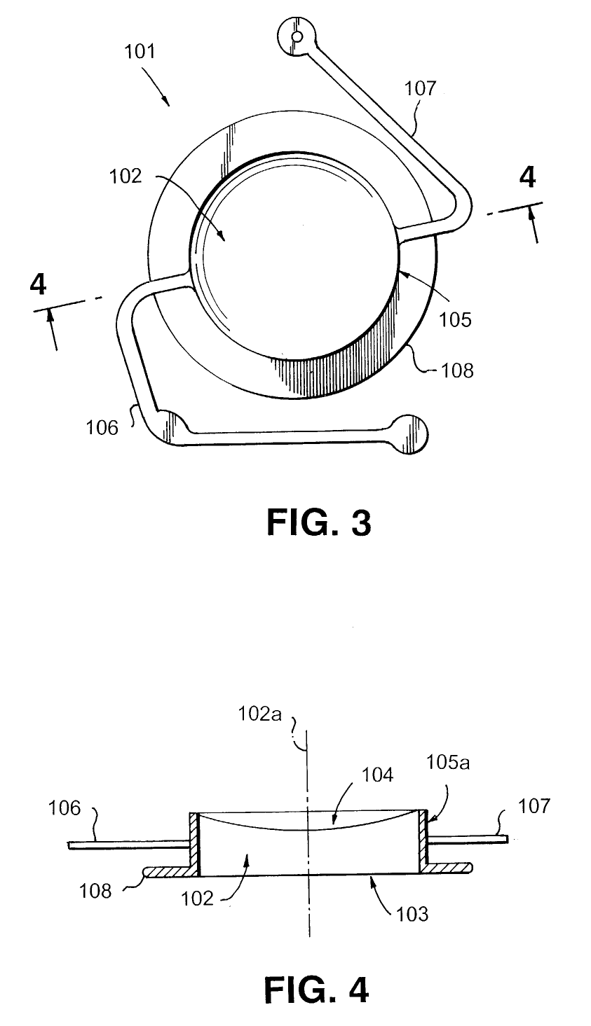

FIGS. 3 and 4 show, respectively, a frontal view and a cross-sectional view of an anterior-chamber intraocular lens 101 according to the invention. The cross-sectional plane of FIG. 4 is defined by the axis B-B of FIG. 3 and the optical axis 102a of the IOL 101. The central portion of the IOL 101 is a transparent body in the shape of a planar/concave optical lens 102, commonly referred to as the optic portion or “optic” of the IOL. In the inserted condition, the planar surface 103 of the optic 102 faces the retina, while the concave surface 104 faces the cornea. In other words, the planar side 103 represents the posterior face, and the concave side 104 represents the anterior face. The cylindrical, radially facing side wall of the optic will be referred to as the circumference 105 of the optic portion. An annular flange 108 is integrally shaped on the optic portion 102 and extends radially outwards from the circumference 105. In contrast to the optic portion 102, which is transparent, the annular flange 108 is substantially impervious to light.

Fixation elements or haptics 106, 107 extend outwards from the optic portion, originating substantially at diametrically opposite points of the circumference 105. As a preferred alternative, the haptics 106, 107 could originate from the flange 108 as an outward continuation or extension of the flange. As in the prior-art IOL of FIGS. 1 and 2, the haptic elements 106, 107 run in partially radial and partially tangential directions, generally in an opposite sense with respect to each other. The haptic elements 106, 107 are designed to hold the lens fixed at three points.

In the IOL of FIGS. 3 and 4, the annular flange 108, the haptic elements 106 and 107, and possibly even the optic portion 102 are designed to be resilient, i.e., springy, such that they can be bent or folded in order to minimize the overall size of the IOL during the insertion process. After implantation in the anterior chamber, the TOL returns to its original undeformed shape shown in FIG. 3 with the haptic elements 106, 107 spread out to position and hold the IOL in its intended position in the anterior chamber.

The circumference 105 has a light-blocking surface 105a to block light beams from entering the optic portion through the circumference. The circumference 105 should further be made non-reflective to light beams that enter the optic portion 102 through the anterior face 104 at an oblique angle, so that they cannot be reflected from the inside of the circumference 105.

The substantially light-impervious and non-reflective properties of the annular flange and of the circumference of the optic portion can be achieved, e.g., by an opaque and non-reflective coating, by a chemical or mechanical treatment, or a combination of different measures. The substantially light-impervious and/or non-reflective areas or portions of the IOL according to the invention achieve the same purpose as the frame 12 of the prior-art IOL in FIGS. 1 and 2, i.e., they prevent stray light from reaching the retina and producing a sensation of glare for the wearer of the IOL. However, unlike a the frame 12, the treated and/or coated circumference does not take up an additional ring-shaped space around the optic, so that the effective, useful diameter of the optic can be maximized to the full extent that is compatible with the dimensions of the anterior chamber.

The integrally shaped TOL 101 can be manufactured as a homogeneous unit using the same material throughout, or it can be a composite where, e.g., the flange portion 108 and/or the haptic elements 106, 107 are made of a different material and connected to the optic portion by an integral bond.

Preferred materials for the IOL of the present invention include hydrophilic materials capable of absorbing and holding an appreciable amount of water, e.g., 20% or more of the “dry weight” of the material. The reasons for choosing hydrophilic materials are that they are tissue-friendly, and at least some of them also allow the design of IOLs that expand and take their final shape by hydrating (absorbing water) after they have been inserted. Suitable hydrophilic materials for use in the IOL 101 include acrylic polymers and hydrogels. However, at least a part of the IOL 101 could also be made of another material such as polymethylmethacrylate, or of a silicone material.

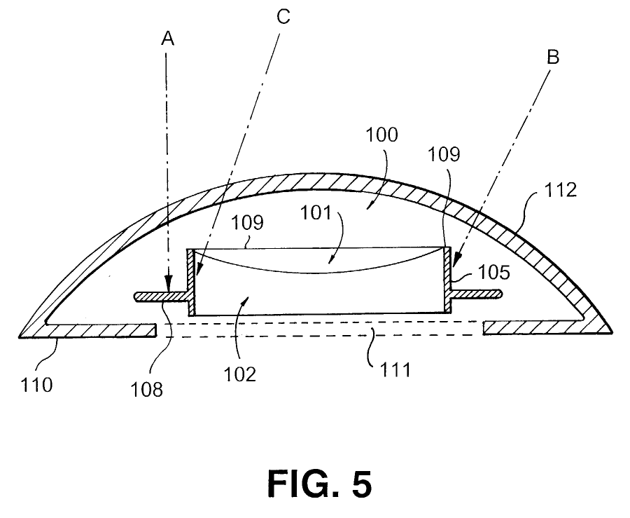

FIG. 5 shows the lens 101 of the present invention positioned inside an anterior chamber 100 in front of a dilated iris 110. An incident light ray A which passes near the peripheral edge 109 of the lens 101 is impeded from reaching the iris aperture 111 because of the presence of the flange 108 which acts as a light barrier. A light ray B entering the eye at an oblique angle and falling on the circumference 105 of the optic 102 is blocked from entering the optic by the opaque coating or surface treatment on the circumference 105. A third kind of incident light ray, C, enters the optic 102 through the anterior face, but due to the oblique angle of incidence, the light ray C falls on the inside of the circumference, where it is captured or absorbed by the non-reflective treatment of the circumference. If the light ray C were reflected at the circumference it would exit the optic through the posterior face and end up on the retina as stray light. As a result of the opacity of the flange 108 and the opaque and non-reflective treatments of the circumference 105, the three types of stray light as exemplified by the light rays A, B, C are prevented from reaching the retina, so that a wearer of the IOL according to the present invention will not experience any irritating glare. As can further be seen in FIG. 5, the optic portion 102 is designed small enough, so that the anterior edge 109 does not touch the inside of the cornea 112.

The IOL 101 according to the present invention is inserted into the anterior chamber 100 by way of a corneal incision. During insertion into the eye, the cross-sectional dimensions of the IOL are minimized by folding some or all of the portions, i.e., the optic 102, flange 108, and haptics 106, 107 in a way that will minimize the size of the corneal incision. If at least part of the IOL is made of a hydrophilic material, the IOL may be designed for insertion in a dry state, which contributes further to a compact configuration of the IOL to facilitate insertion through a minimal incision. Once the IOL 101 is inserted into the eye, it takes on its intended shape by unfolding and/or by absorbing fluid and thereby expanding.

As mentioned previously, minimizing the size of the incision is a significant concern since, understandably, the smaller the corneal incision size the less trauma experienced by the patient, and in turn, the less the pain and discomfort endured then and thereafter, not only because of the incision itself but also because of the number and/or size of any needed sutures.

While the invention has been shown and described with reference to a preferred embodiment thereof, it will be understood by those skilled in the art that various changes in form and details may be made therein without departing from the spirit and scope of the invention.

What is claimed is:

1. A high-myopia intraocular lens for surgical implantation in an anterior chamber of an eye, said intraocular lens being constructed as a single, integral unit, comprising:
a substantially circular and substantially transparent optic portion with a concave anterior face, a planar posterior face, and a substantially cylindrical circumference, said cylindrical circumference being substantially impervious to incident light falling on said circumference from outside the optic portion and non-reflective to incident light falling on said circumference from inside the optic portion;
an annular flange portion extending substantially in a plane in an outward radial direction from the optic portion, said flange portion being substantially impervious to light and further having an outside diameter at least equal to an aperture diameter of a dilated pupil of the eye;
haptic elements extending substantially in an outward radial direction from the peripheral area and serving to fixate the intraocular lens in the anterior chamber,
wherein at least the annular flange portion and the haptic elements are sufficiently flexible to be folded towards the center during the surgical implantation and to return to an unfolded state after the surgical implantation.

2. The intraocular lens of claim 1, wherein the optic portion, likewise, is sufficiently flexible to be folded during the surgical implantation and to return to an unfolded state after the surgical implantation.

3. The intraocular lens of claim 1, wherein the optic portion has a size and shape adapted to avoid contact between the optic portion and an inner corneal surface when the intraocular lens is in an implanted state.

4. The intraocular lens of claim 1, wherein the intraocular lens comprises a hydrophilic material.

5. The intraocular lens of claim 4, wherein the hydrophilic material is capable of holding a water content of at least 20 percent.

6. The intraocular lens of claim 5, wherein the hydrophilic material is capable of holding a water content from 20 percent to 30 percent.

7. The intraocular lens of claim of claim 4, wherein the hydrophilic material is an acrylic polymer.

8. The intraocular lens of claim 4, wherein the hydrophilic material is a hydrogel.

9. The intraocular lens of claim 4, wherein the intraocular lens is implanted in the eye in a non-hydrated state and expands after implantation by absorbing fluid.

10. The intraocular lens of claim 1, wherein the intraocular lens comprises polymethylmethacrylate.

11. The intraocular lens of claim 1, wherein the intraocular lens comprises a silicone material.

12. The intraocular lens of claim 1, wherein not all of the optic portion, the flange portion and the haptic elements are made of identical material and said single, integral unit comprises at least one integral bond between portions made of different materials.

13. The intraocular lens of claim 1, wherein the optic portion has a diameter between 3.5 and 5.0 millimeters and the flange portion extends radially outwards from the optic portion by 0.3 to 1.0 millimeters.

14. The intraocular lens of claim 1, wherein at least one of the flange portion and the circumference is substantially impervious to light as a result of a chemical treatment.

15. The intraocular lens of claim 1, wherein at least one of the flange portion and the circumference is substantially impervious to light as a result of a mechanical treatment.

16. The intraocular lens of claim 1, wherein at least one of the flange portion and the circumference is substantially impervious to light as a result of a surface coating.

17. The intraocular lens of claim 1, wherein the circumference is non-reflective as a result of a chemical treatment.

18. The intraocular lens of claim 1, wherein the circumference is non-reflective as a result of a mechanical treatment.

19. The intraocular lens of claim 1, wherein the circumference is non-reflective as a result of a surface coating.

20. The intraocular lens of claim 1, wherein the flange portion extends in a plane intermediate and parallel to the anterior and posterior faces, said plane being positioned closer to the posterior face than to the anterior face.
